(19) United States
(12) Patent Application Publication
(10) Pub. No.: US 2015/0133938 A1
(43) Pub. Date: May 14, 2015

(54) FIXING DEVICE FOR FIXING THE FRACTURE ENDS OF THE BONES IN A BONE FRACTURE, AND TENSIONING ELEMENT, ANGLE CLAMPING SLEEVE AND CORRESPONDING METHOD

(71) Applicant: Hipp medical AG, Kolbingen (DE)

(72) Inventor: Markus Waizenegger, Kolbingen (DE)

(21) Appl. No.: 14/407,267

(22) PCT Filed: Mar. 13, 2013
(86) PCT No.: PCT/EP2013/000750

§ 371 Date: Dec. 11, 2014

(30) Foreign Application Priority Data
Jun. 13, 2012 (DE) 102012105123.2

(51) Int. Cl. A61B 17/82

(52) U.S. Cl. CPC A61B 17/82
USPC 606/74

(57) ABSTRACT

The invention proposes a fixing device for fixing the fracture ends of bones in a bone fracture comprising at least two tensioning elements (2a, 2b), each of which has a contoured body that is hollow when viewed from above, has a circumferential wall (4) and two face sided receiving sections (6a, 6b) lying opposite to each other, at least one clamping sleeve and at least two fixing means which can be position in the face sided receiving sections. The at least two tensioning elements (2a, 2b) are positioned in relation to each other in such a way that one receiving section (6a, 6b) of each tensioning element (2a, 2b) is aligned with the respective other receiving section and are penetrated by the clamping sleeve in such a way that both tensioning elements (2a, 2b) are coupled to each other. According to invention the clamping sleeve furthermore is an angle clamping sleeve (15) which is connected with the at least two tensioning elements (2a, 2b) in such a way that the latter lie in a predetermined angular position in relation to each other.

The invention relates to a fixing device for fixing the fracture ends of the bones in a bone fracture according to claim 1 as well as to tensioning element used therefore according to claim 23 and an angle clamping sleeve used therefore according to claim 25. The invention also relates to a method for fixing fracture ends of bones in a bone fracture according to claim 27.

When a bone fracture is immobilized insufficiently, a callus formation, i.e. a tyloma-like thickening of the ends of the fracture from overgrowing bone tissue, can occur. In order to avoid such an indirect fracture healing via a callus, bone plates are used which are applied and attached to the outer surface of a broken bone so that the fracture site is set, i.e. fixed, during the healing process.

In addition, the healing process of a fracture is favorably influenced when a compression is applied on the jointed site of fracture. In this way, a particularly close adaptation is caused, i.e. a low clearance by which the broken ends grow to each other again.

US 2009/0036930 A1 discloses a bone fixing device for fixing the fracture ends of the bones in a bone fracture under compression during the healing process. A modular fixing system can be formed by stringing together multiple bone fixing devices. However, the multiple bone fixing devices of the modular fixing system cannot be positioned to each other in a certain orientation prior to the insert of the fixing means. Here, a positioning of the bone fixing devices oriented to each other is only possible after inserting the fixing means in the particular fracture ends of the bones.

Further, from DE 20 2011 109808 U1 is known a tensioning element which is composed of a contoured body designed as a circumferential wall with resilient angled sections and receiving sections. In this case, also a plurality of tensioning elements can be connected by openings and cut-outs in the receiving sections in the sense of a tongue and groove connection. By connecting a plurality of tensioning elements a modular configuration is provided, by which a fixing device can be individually adapted to the structure of a fracture and the local anatomic conditions. As in the case of the above-mentioned prior art, it reveals as a disadvantage of the discussed tensioning element that the connected tensioning elements cannot be firmly positioned to each other prior to the pushing through of the fixing means through the adjacent tensioning elements and the subsequent inserting of the fixing means in the fractures ends. The adaption to the structure of a fracture and the local anatomic conditions can occur during an operation not until the direct fixing of the tensioning elements at the bones.

Thus, it is object of the present invention to provide a fixing device having a firm orientation of a plurality of tensioning elements even before fixing the individual tensioning elements at the fracture ends of the bones.

This object is solved by a fixing device with the features of claim 1.

According to the invention a fixing device for fixing the fracture ends of bones in a bone fracture comprising at least two tensioning elements, each of which has a contoured body that is hollow when viewed from above, has a circumferential wall and two face sided receiving sections lying opposite to each other, at least one clamping sleeve and at least two fixing means which can be position in the face sided receiving sections. The at least two tensioning elements are positioned in relation to each other in such a way that one receiving section of each tensioning element is aligned with the respective other receiving section and are penetrated by the clamping sleeve in such a way that both tensioning elements are coupled to each other. According to invention the clamping sleeve furthermore is an angle clamping sleeve which is connected with the at least two tensioning elements in such a way that the latter lie in a predetermined angular position in relation to each other.

In this way the tensioning elements can be oriented in a predetermined angle particularly well beforehand, e.g. on the basis of information and findings of prior examinations, and prior to the inserting of the fixing means in the fracture ends.

The adaption of the tensioning elements to the structure of the fracture and the local anatomic conditions thus is possible prior to the direct fixing of the tensioning elements at the bones. By means of imaging methods, for example conventional x-ray tomography, computer tomography (CT), magnetic resonance imaging (MRI), positron emissions tomography (PET) or the same, the structure of the fracture can be analyzed and the angular position of the tensioning elements fixing the fracture ends of the bones can be oriented in advance. Accordingly, a predetermined fixing device can be configured as tensioning element modular system.

Especially in complex fractures, which require an extensive preparation, the individual tensioning elements can be oriented to each other and assembled outside of the body in a predetermined angular position in accordance to the break line of the fracture. Thereby, the physical burden on the patient can be considerably reduced.

If a multitude of tensioning elements is assembled to a fixing device prior to the operation, this results in a saving of time during the subsequent operation, whereby the burden of the patient is further reduced. Because of the shorter operation, the patient needs lesser amounts of anesthetics.

By preparing the fixing device prior to the operation, the risk of error of injuring the patient or assembling the fixing device in a wrong way is reduced. The fixing device assembled beforehand can pass one or more quality tests which can be performed by trained specialists. A complex quality test during the operation, on the other hand, is very difficult because of the limited time period and the limited space available. On the other hand, the tensioning elements can be assembled from the doctors peacefully without time pressure, which also reduces the risk of error.

Although two fixing means are used above, three fixing means can also be used to improve the fixing of the fixing device to the bone.

Apart from the orientation of the tensioning elements in a predetermined angular position, the fixing device is also able to align the at least two adjacent tensioning elements with respect to their longitudinal axes.

Further embodiments of the fixing device of the present invention are subject-matter of dependent claims 2 to 22.

Further, the angle clamping device can be connected in a form fitting manner to the at least two tensioning elements. By the form fit constructed by the angle clamping device and the at least two tensioning elements, a particular well locking is provided.

The form fit can be performed in such a way that at the inner surface of at least one of the face sided receiving sections of the tensioning element is formed at least one protrusion and/or at least one recess and that at an outer surface of the angle clamping sleeve is formed at least one protrusion and/or at least one recess. The protrusions and recesses of the tensioning elements are formed in a way corresponding to the protrusions and recesses of the angle clamping sleeve. In this way a particular easy form fit can be provided.

Particular good results however appear if along the inner surface of the face sided receiving section of the tensioning element at least in parts internal teeth are formed and if along the outer surface of the angle clamping sleeve at least in parts external teeth are formed. The internal teeth of the tensioning elements are formed in a way corresponding to the external teeth of the angle clamping sleeve. The finer the partition of the particular toothing is chosen, the smaller the possible angular steps when locking the at least adjacent tensioning elements.

Moreover, at least one of the face sided receiving sections can be formed tapered, in particular conical. In this configuration during the inserting of the fixing means, in particular of a cortical screw having a screw head corresponding to the receiving section a stabilization of the angle regarding the angle between the fixing means and the tensioning element can be advantageously achieved. The tapered configuration of the face sided receiving section encompasses also its protrusions, recesses and internal teeth respectively.

Furthermore, the at least two tensioning elements can be coupled to each other via the face sided receiving sections by a tongue and groove connection. In this way the tensioning elements are coupled to each other in a reliable and easy way. With this configuration a tensioning device modular system can be provided especially well.

Moreover, the angle clamping sleeve can be connected in a force-fitted manner with the at least two tensioning elements. The surfaces of the angle clamping sleeve and the tensioning elements interacting with each other can be conditioned in such a way that a particular good frictional connection is possible. Besides, the angle clamping sleeve can be configured in such a way that it presses in such a way against the adjacent tensioning devices that the tensioning devices are locked with respect to the angular position to each other by friction. The angle clamping sleeve thereby can comprise a larger outer circumference than the aligned area of the tensioning elements.

In order to further enhance the angular stabilization between multiple fixing means, in particular cortical screws, and the tensioning element, a thread can be formed on an inner surface of the angle clamping sleeve. Hereby, the angle clamping sleeve and the tensioning elements constitute an even better operative connection regarding the angle stabilization.

A further possibility is to form the angle clamping sleeve from a material which allows screwing in a thread in its inner surface by the fixing means. The fixing means thereby comprises the corresponding structure of the inner surface of the angle clamping sleeve. In this way, when the fixing means is inserted into the fracture ends of the bones, at the same time a thread is screwed into the inner surface of the angle clamping device. The angle stabilization thereby is significantly higher than in comparison to a thread tapped beforehand into the inner surface of the angle clamping sleeve because there are no free play and clearance between both of the components.

Further, the inner surface and/or the outer surface, and external teeth respectively, of the angle clamping sleeve can be formed at least in parts tapered, in particular conical. This measurement also enhances the angle stability between bone plates and fixing means. In this tapered configuration of the inner surface it can also be understood that the thread screwed into the inner surface is formed in a tapered way so that a tapered thread exists. Although an external teeth is mentioned above, it is also possible that a single protrusion or a plurality of protrusions are formed in a tapered way.

In order to insert the angle clamping sleeve in a simple way into the aligned region made by the at least two adjacent tensioning devices, the angle clamping sleeve can provide a continuous slit along its longitudinal axis. The angle clamping sleeve therefore is compressible with respect to its circumference, whereby the circumference is reduced at least in such a way that it can be easily inserted into the aligned region of the at least two adjacent tensioning elements. Although in this connection inserting the angle clamping sleeve into the aligned region is mentioned, it is also possible the individual tensioning elements can be positioned on the angle clamping sleeve. If a fixing means in inserted, which comprises a larger circumference than the inner circumference of the angle clamping sleeve, the continuous slit furthermore causes that the angle clamping sleeve is deformed outwardly. In this way a spreading of the sleeve can be generated, which affects the tensioning elements and therefore enhances the firm orientation of the adjacent tensioning elements in a predetermined angular position.

In order to enhance the spreading effect of the angle clamping sleeve during the fixing to the fracture ends of the bone, the angle clamping sleeve can comprise a non-continuous slit along its longitudinal axis. The continuous slit and the at least one non-continuous slit can be positioned in the circumferential direction in a predetermined distance or in a non-predetermined distance.

Alternatively, the angle clamping sleeve can comprise a lateral surface groove. This lateral surface groove takes, on the one hand, the bending stress occurring between adjacent tensioning elements and, on the other hand, the stress introduced by a deformation of the angle clamping device. In a preferred way therefore a reduction of stresses within the fixing device is possible.

Furthermore, the angle clamping sleeve can comprise at least one at least in parts circumferential crank. If the angle clamping sleeve is positioned on the surface of the bone with the crank, the angle clamping sleeve can house the tensioning elements and space same from the bone. The spacing of the tensioning elements from the bones avoids that the thin-walled wall of the tensioning elements injures the surface of the bones. Further, the formed crank enables that the angle clamping sleeve does not slip through when the angle clamping sleeve is inserted into the aligned region of the tensioning elements.

The angle clamping sleeve furthermore can be configured with a flap, by which a locking ring retains the angle clamping sleeve with respect to at least one tensioning element. By the crank formed at one end of the angle clamping sleeve and the flap formed at the other end of the angle clamping sleeve together with the locking ring, the adjacent tensioning elements can be rigidly connected to each other in the height direction.

Moreover, the angle clamping sleeve can be deformable in such a way that the angle clamping sleeve during the inserting of the angle clamping sleeve can be compressed initially and thus the inserting can be facilitated. The angle clamping sleeve, however, can also get spread after or during the insertion of the fixing means and therefore further enhancing the rigid orientation of the angular position between the tensioning elements. The deformation can be elastic or a plastic whereby the plastic deformation is desired not until the fixing means have been inserted.

Further, the angle clamping sleeve can be divided in radial direction into two sections and the radial inner section or the radial outer section can be made of an absorbable synthetic material. The absorbable synthetic material is a material which is degradable by the surrounding body tissue. Absorbable synthetic materials, however, have relative brittle material properties. They therefore are suitable only to a limited extend for area which are exposed to mechanical loads. In addition, such materials are not provided for generating elastic bodies. By the absorption of one of the two sections a clearance arises between the fixing means and the tensioning elements resulting in force isolation between the fixing means. If in a preferred way the outer section is made of a metal and the inner section is made of an absorbable synthetic material, a localization of the tensioning elements remains by the cranks of the outer section. In this way, a release of the tensioning element from its arrangement and a possible impairment of the adjacent body tissue can be prevented.

Furthermore, a fixing means can be pushed through the angle clamping sleeve. Accordingly, the tensioning elements are not only connected to each other and oriented to each other in a predetermined angular position but also inserted into and fixed to the bone in the aligned region by a fixing means. The fixing means can be a conventional cortical screw.

In addition, the cortical screws can comprise a screw head provided with a thread. In this way an angle stabilization between the cortical screws, in particular in the region of the screw head, and the at least one tensioning element can be achieved. The screw head hereby can be configured as circular keying profile, Phillips head, slotted, Torx, Allen key or the same.

Regarding the mentioned angle stabilization it has been proven to be particularly positive that the screw head of the cortical screws comprises a thread pitch different from the thread pitch of the screw shaft. If the screw head of a cortical screw has a thread pitch smaller than the thread pitch of the screw shaft, the operative connection between the cortical screw, in particular the screw head, and the angle clamping sleeve can be enhanced because of the additional entanglements and/or the mechanical deformations of the angle clamping sleeve.

An additional improvement of the angle stability is that the screw head of the cortical screw is formed tapered, in particular conical. By a configuration of the screw head as a circular cone, the forces transferred by the cortical screw are transferred to the tensioning element particular well.

According to a further aspect of the present invention according to claim 23 a tensioning element, in particular for a fixing device, comprising a contoured body that is hollow when viewed from above, has a circumferential wall and two face sided receiving sections lying opposite to each other, is proposed. An inner surface of at least one of the receiving sections is configured in such a way that the tensioning element is locked in a predetermined angular position with respect to a further tensioning element in a form fitting manner or in force fitting manner.

Regarding the advantages of the tensioning element of the present invention reference is made to the accordant above-mentioned remarks.

Preferred embodiments of the method of the present invention are subject-matter of dependent claim 24. To this reference is also made to the above-mentioned accordant advantages.

According to a further aspect of the present invention according to claim 25 an angle clamping sleeve, in particular for a fixing device, comprising means for establishing a form fit or a force fit at an outer surface so that at least two tensioning elements are lockable in a predetermined angular position, is proposed.

Regarding the advantages of the angle clamping sleeve of the present invention reference is made to the accordant above-mentioned remarks.

Preferred embodiments of the method of the present invention are subject-matter of dependent claim 26. To this reference is also made to the above-mentioned accordant advantages.

According to a further aspect of the present invention according to claim 27 a method for fixing the fracture ends of the bones in a bone fracture is proposed, comprising the following steps: positioning at least two tensioning elements in a predetermined angle in such a way that a respective receiving section of the at least two tensioning elements are aligned to each other; inserting of an angle clamping sleeve into the aligned region of the at least two tensioning elements in such a way that a form fitting connection is formed so that the at least two tensioning elements are angle stable with respect to their longitudinal axis; pushing through of a fixing means through the angle clamping sleeve; and inserting the fixing means pushed through the angle clamping sleeve into the bone.

Regarding the advantages of the method for the fixing device of the present invention reference is made to the accordant above-mentioned remarks.

Exemplary preferred embodiments of the method of the present invention are subject-matter of the dependent claims 28 and 29.

Further, in the method for fixing the fracture ends of the bones in a bone fracture the step of pushing through of a fixing means through the angle clamping sleeve can be carried out prior to the step of inserting a angle clamping sleeve into the aligned region.

Alternatively, in the method for fixing the fracture ends of the bones in a bone fracture a deformable angle clamping sleeve can be used so that by the step of inserting the fixing means into the bone compressive forces are transferred to a inner surface of the angle clamping sleeve so that the deformable angle clamping sleeve with its outer surface is furthermore pressed against the face sided receiving sections and the angle stability of the longitudinal axes of the at least two tensioning elements is increased.

The features and functions of the present invention described above as well as the further aspects and features will be described in the following by a detailed description of preferred embodiments under reference to the attached figures. In this connection shows:

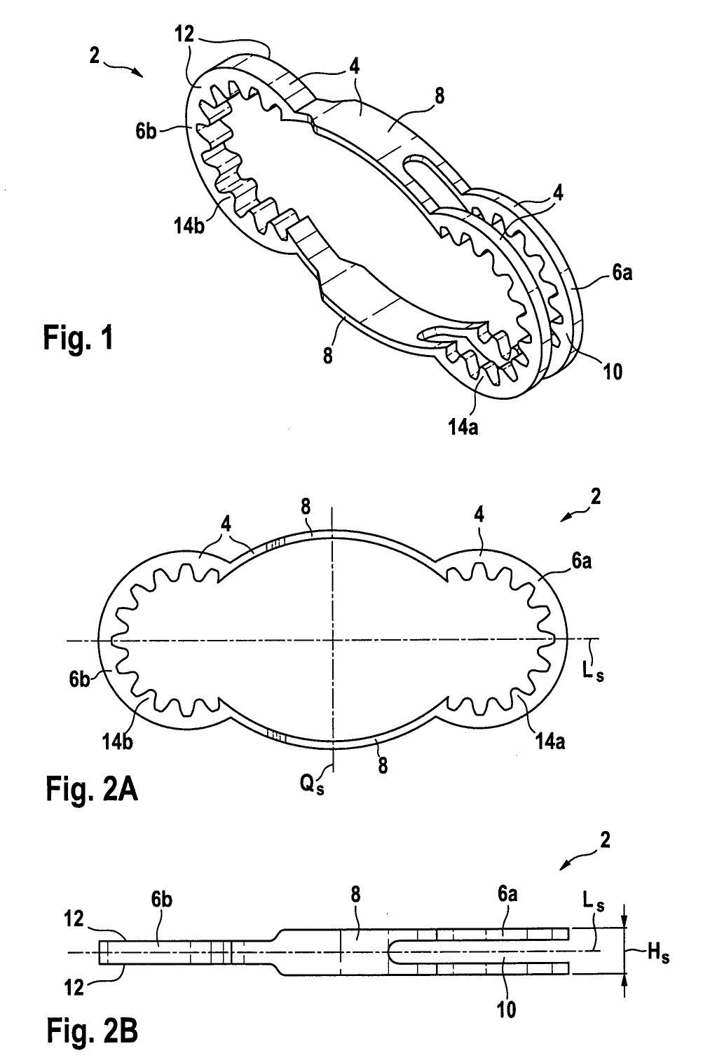

FIG. 1 a perspective view of a first embodiment of a tensioning element of the present invention;

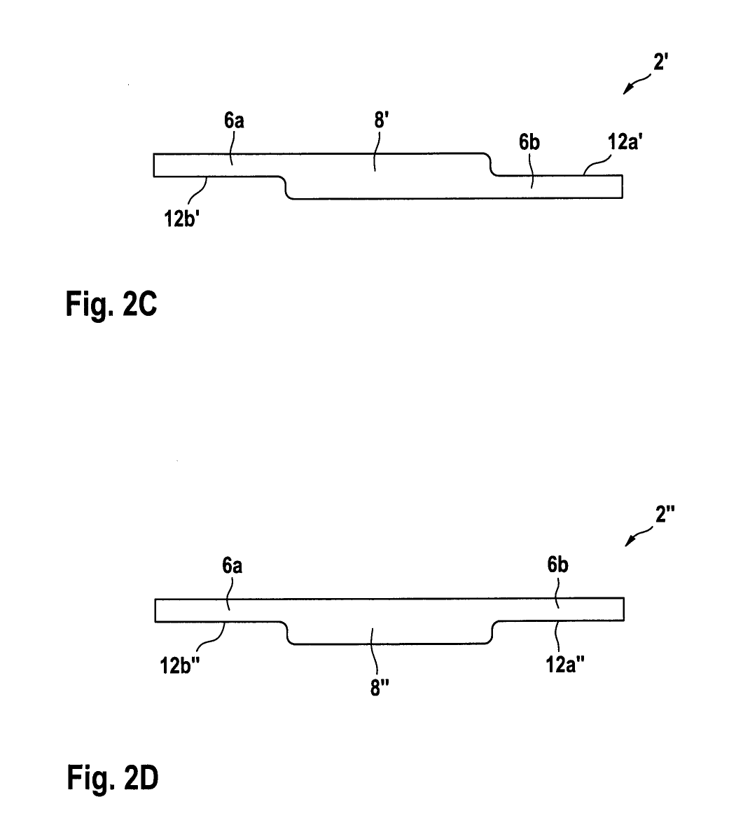

FIG. 2A a top view of the first embodiment of the tensioning element of the present invention according to FIG. 1;

FIG. 2B a side view of the first embodiment of the tensioning element of the present invention according to FIG. 1;

FIG. 2C a side view of a second embodiment of an alternative tensioning element;

FIG. 2D a side view of a third embodiment of an alternative tensioning element;

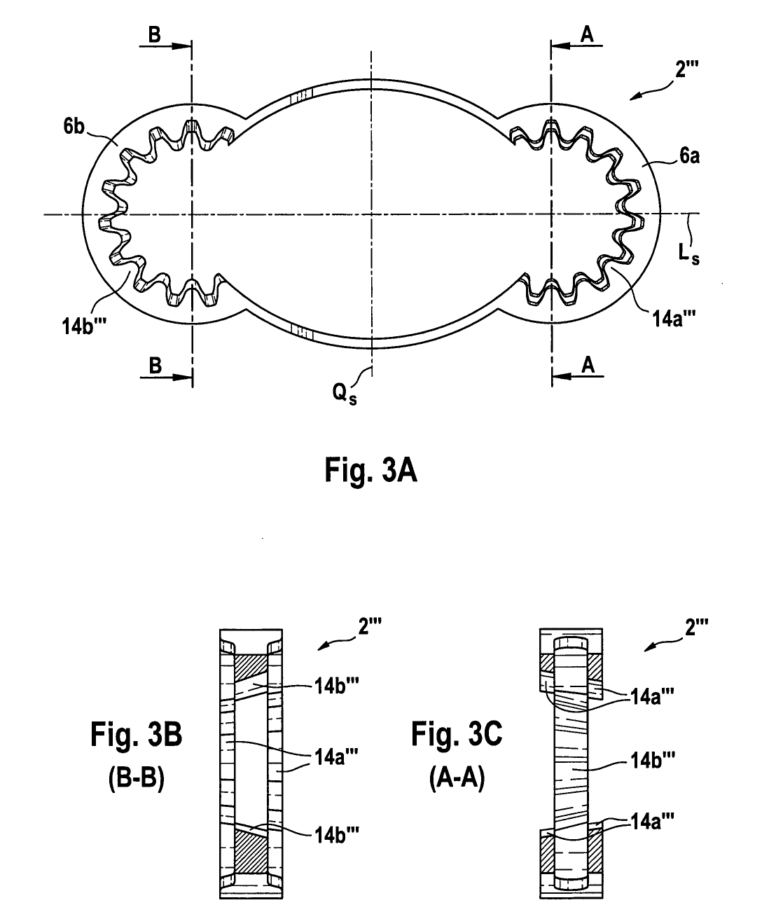

FIG. 3A a top view of a fourth embodiment of a tensioning element of the present invention;

FIG. 3B a sectional view of section B-B of the fourth embodiment of the tensioning element of the present invention according to FIG. 3A;

FIG. 3C a sectional view of section A-A of the fourth embodiment of the tensioning element of the present invention according to FIG. 3A;

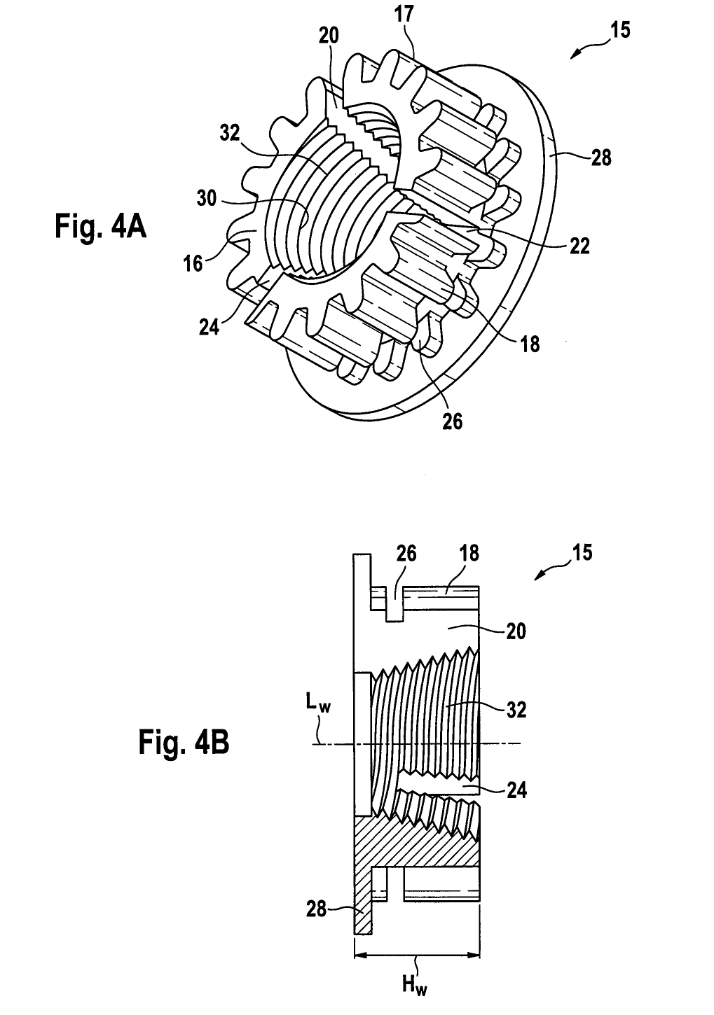

FIG. 4A a perspective view of a first embodiment of an angle clamping sleeve of the present invention;

FIG. 4B a sectional view of the first embodiment of the angle clamping sleeve of the present invention according to FIG. 4A;

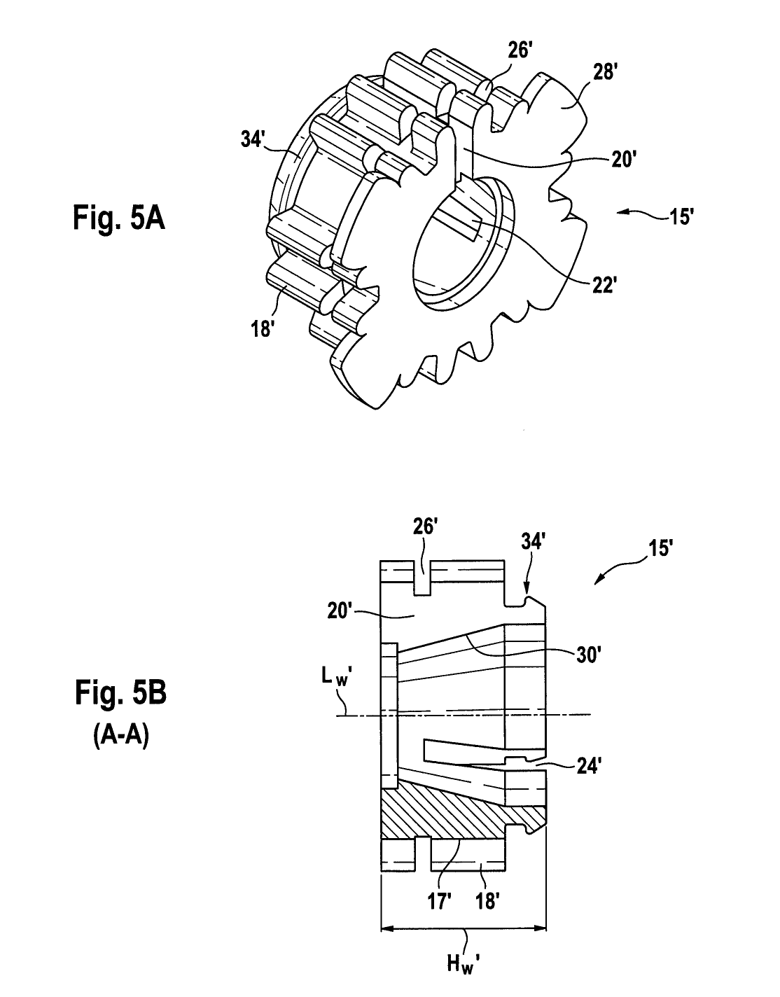

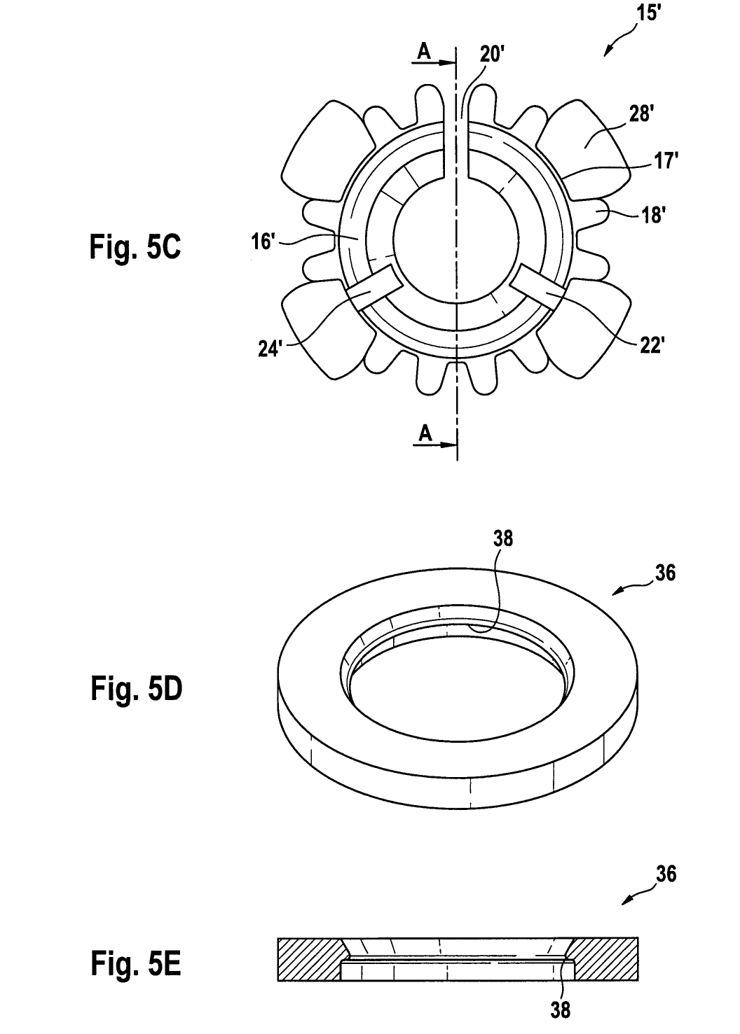

FIG. 5A a perspective view of a second embodiment of an angle clamping sleeve of the present invention;

FIG. 5B a sectional view of the second embodiment of the angle clamping sleeve of the present invention according to FIG. 5A;

FIG. 5C a top view of the second embodiment of the angle clamping sleeve of the present invention according to FIG. 5A;

FIG. 5D a perspective view of a locking ring;

FIG. 5E a sectional view of the locking ring according to FIG. 5D;

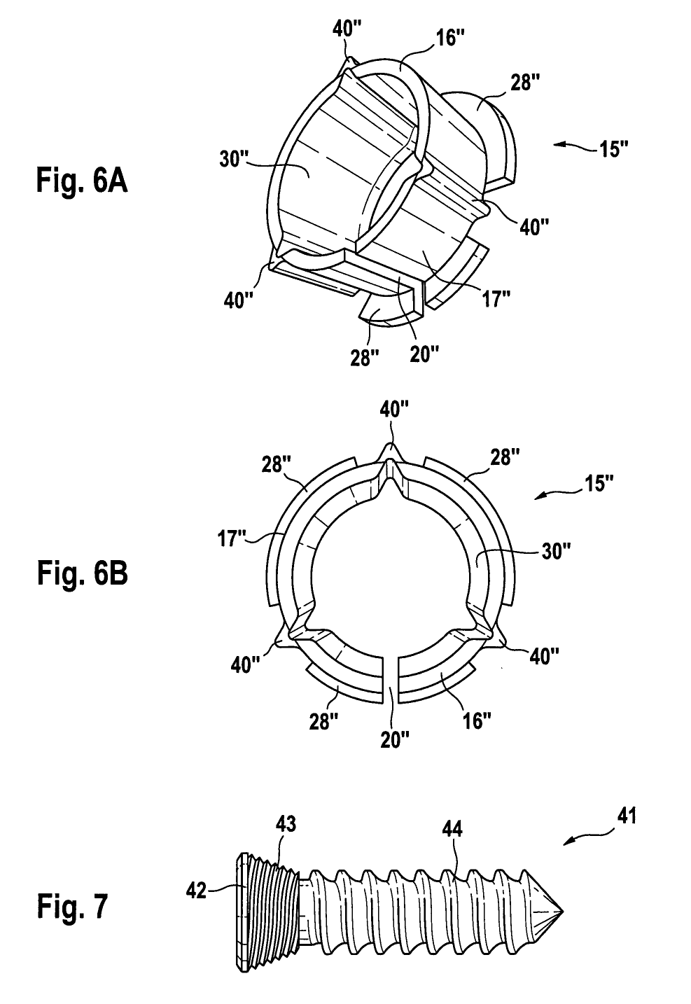

FIG. 6A a perspective view of a third embodiment of an angle clamping sleeve of the present invention;

FIG. 6B a top view of the third embodiment of the angle clamping sleeve of the present invention according to FIG. 6A;

FIG. 7 a side view of a cortical screw;

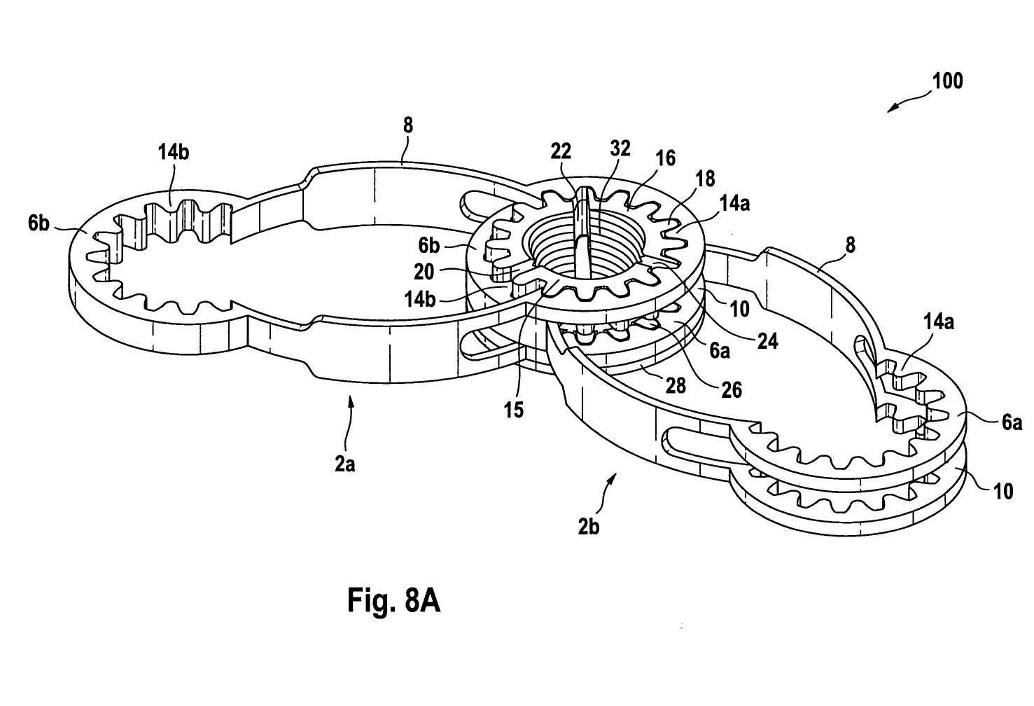

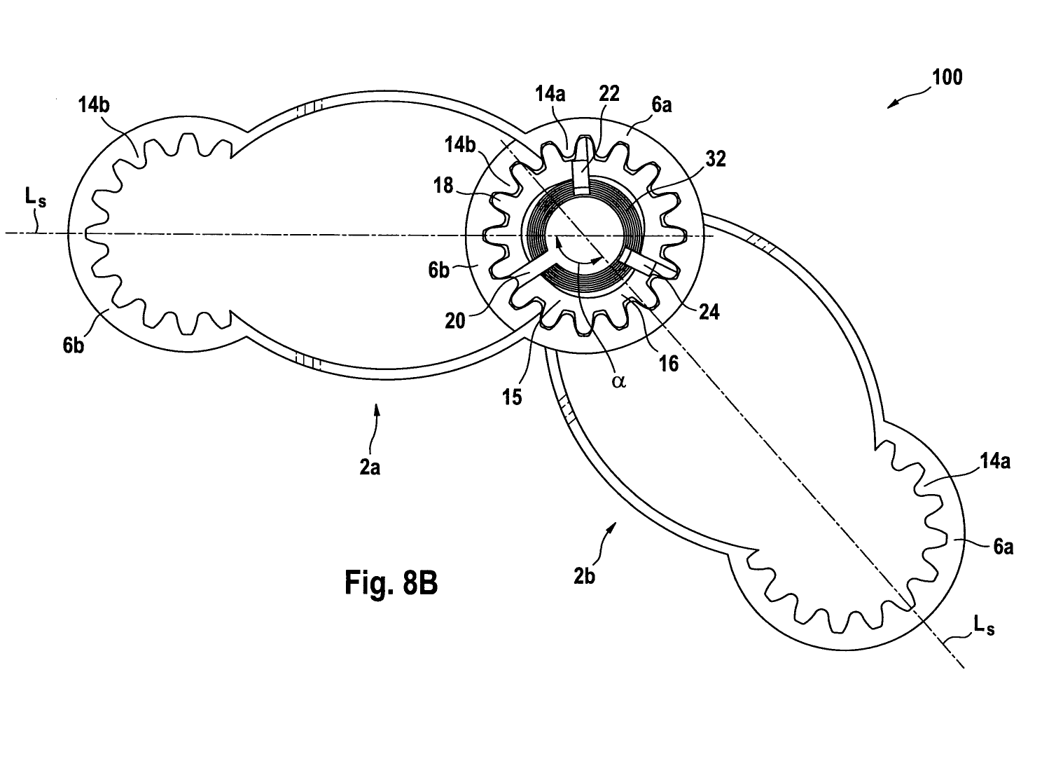

FIG. 8A a perspective view of an embodiment of fixing device of the present invention; and

FIG. 8B a top view of the embodiment of the fixing device of the present invention according to FIG. 8A.

Before the mode of operation of the fixing device 100 is described in FIGS. 8A and 8B, first of all the individual components are described.

FIG. 1 is a perspective view and FIG. 2A is a top view of a first embodiment of a tensioning element 2 of the present invention. The tensioning element 2 is composed of a contoured body which is hollow when viewed from above and has a circumferential wall 4. The tensioning element 2 further comprises two face sided receiving sections 6a, 6b lying opposite to each other and two lateral flanks with angled sections 8. The two face sided receiving sections 6a, 6b lie opposite to each other with respect to the transverse axis Qₛ of the tensioning element 2 and comprise a curvature directing outwards. The two angled sections 8 lie opposite to each other with respect to the longitudinal axis Lₛ of the tensioning element 2 and comprise a curvature directing outwards. The tensioning element 2 shown in FIG. 1 is formed basically rhombic.

The wall 4 of the tensioning element 2 comprises an opening 10 in the region of the face sided receiving section 6a and in parts in the lateral flanks as well as cut-outs 12 in the region of the face sided receiving section 6b and in parts in the lateral flanks. Such configuration can clearly be seen in the side view of FIG. 2B. The opening 10 and the cut-outs 12 are formed in a way corresponding to each other so that by such a tongue shape and/or such a groove shape of wall sections a complementary fitting is provided, by which the contours of adjacent tensioning elements 2 can be brought into intersection. In FIG. 2B furthermore the height Hₛ of the tensioning element 2 is depicted. Although the tensioning element 2 comprises cut-outs 12 and an opening 10, the wall shown in FIGS. 1, 2A and “B represents a circumferential wall 8.

Further, the opening 10 and the cut-outs 12, different from the first embodiment, can be formed solely in the region of the receiving sections 6a, 6b.

The face sided receiving sections 6a, 6b each comprise an inner teeth 14a, 14b. The face sided receiving section 6a and its inner teeth 14a are divided by the opening 10 into two parts, wherein the individual teeth of the inner teeth 14a are located opposite to each other when viewing the top view of FIG. 2a.

As an alternative to the opening 10 and the cut-outs 12 of the first embodiment, in FIGS. 2C and 2D a second and a third embodiment of the tensioning elements 2′, 2″ are shown. The cut-outs 12a′ 12′ of the alternative tensioning element 2′ in FIG. 2C lie diametrically opposite to each other in the accordant receiving sections 6a, 6b when viewing the side view. The cut-outs 12a″ and 12b″ of the alternative tensioning element 2″ in FIG. 2D lie in a minor image to each other in the accordant receiving sections 6a, 6b when viewing the side view. The embodiments shown in FIGS. 2C and 2D of the face sided receiving sections 6a, 6b of the tensioning elements 2′, 2″ also enable the coupling of several tensioning elements 2′, 2″ to a fixing device 100.

FIG. 3A is a top view of a fourth embodiment of the tensioning element 2″′ of the present invention, whereby FIGS. 3B and 3C are sections B-B and A-A from FIG. 3A. The tensioning element 2″′ is configured similar to the tensioning element 2 of FIGS. 1, 2A and “B. The only difference is that the inner teeth 14a′″, 14b′″ is formed in a conically tapered way, as shown in FIGS. 3B and 3C. The arrangement of the tensioning element 2 with the tapered inner teeth 14a″′, 14b″′ takes places in such a way that the tapered section is directed to the surface of the bone, in order to achieve an improved angle stability when the fixing means are inserted.

FIG. 4A is a perspective view and FIG. 4B is side view of a first embodiment of an angle clamping sleeve 15 of the present invention. The angle clamping sleeve 15 comprises a circumferential external teeth 18 on its outer surface 17, which extends over the height H_{w} of the angle clamping sleeve 15. The wall 16 of the angle clamping sleeve 15 therefore is formed integrally with the external teeth. The wall 16 and the external teeth 18 are interrupted by a continuous slit 20 and two non-continuous slits 22, 24. The continuous slit 20 as well as the non-continuous slits 22, 24 run in the direction of the longitudinal axis L_{w} of the angle clamping sleeve 15.

The external teeth 18 furthermore is interrupted by a lateral surface groove 26 which extends transversally to the longitudinal axis L_{w} of the angle clamping sleeve 15. The lateral surface groove 26 is provided, on the one hand, in order to absorb bending stress occurring between two adjacent tensioning elements. On the other hand, stresses occurring when spreading the angle clamping sleeve 15 can be absorbed. FIG. 4B shows that the lateral surface groove 26 is not centrally arranged with respect to the height H_{w} of the angle clamping sleeve 15. In this way bending stresses can be adsorbed more effectively.

A circumferential crank 28 is formed in the angle clamping sleeve 15 at one end with respect to the longitudinal axis L_{w} of the angle clamping sleeve 15. The angle clamping sleeve 15 can be inserted into an aligned region of several tensioning elements is such a way that the crank 28 is directed to the bone. Thus, the crank 28 forms a support for the tensioning element 2 and secures the bone. Moreover, the crank 28 can serve to prevent that the angle clamping sleeve 15 does not slip through when the angle clamping sleeve 15 is inserted into the aligned region of the tensioning elements 2.

Although the lateral surface groove 26 is formed with respect to the height H_{w} of angle clamping sleeve 15 closer to the end comprising the crank 28, the lateral surface groove 26 can also be located centrally or in the region of the end being opposite to the crank 28.

A thread 32 is formed on an inner surface 30 of the angle clamping sleeve 15. Said thread 32 is tapered conically in the direction of the crank 28 and servers for angle stabilization between the screwed in fixing means and the tensioning element.

FIG. 5A is a perspective view, FIG. 5B is a side view and FIG. 5C is a top view of a second embodiment of an angle clamping sleeve 15′ of the present invention. The angle clamping sleeve 15′ comprises a partial circumferential external teeth 18′ on its outer surface 17′. A wall 16′ of the angle clamping sleeve 15′ therefore is integrally formed with the external teeth 18′. The wall 16′ and partially the external teeth 18′ are interrupted by a continuous slit 20′ and two non-continuous slits 22′, 24′. The slits 20′, 22′, 24′ run in the direction of the longitudinal axis L_{w} of the angle clamping sleeve 15′.

A partial circumferential crank 28′ is formed at the free end of the angle clamping sleeve 15′. Near the partial circumferential crank 28′ a lateral surface groove 26′ is formed transversally to the longitudinal axis L_{w}′ for adsorbing bending stresses.

At the end opposed to the crank 28′ a circumferential flap 34′ is formed. This flap 34′ serves for retaining the angle clamping sleeve 15′ with respect to the tensioning element 2 via a locking ring 36 described in the following.

A inner surface 30′ of the angle clamping sleeve 15′ comprises a fixing means, is flat and in parts tapered conically.

The angle clamping sleeve 15′ is positioned on the surface of the bone in such a way that the circumferential crank 28′ faces the surface of the bone. The crank 28′ absorbs takes several tensioning elements and saves the sensitive surface of the bone with respect to the thin face sided wall 4 of the tensioning elements. After placing the tensioning elements on the angle clamping sleeve 15′ a locking ring 36 is fixed to the flap 34′ by the projection 38.

FIG. 5D is a perspective view and FIG. 5 E is a sectional view of the locking ring 36. The locking ring 36 comprises an inner circumference and an outer circumference. In the region of the inner surface of the locking ring 36 a projection corresponding to the flap 34′ is formed in order to enable a locking.

FIG. 6A is a perspective view and FIG. 6B is a top view of a third embodiment of an angle clamping sleeve 15″ of the present invention. The angle clamping sleeve 15″ comprises a wall 16″, which on its outer surface 17″ comprises three protrusions 40 being formed in a predetermined distance from each other with respect to the circumference. The protrusions 40″ are spaced from each other by an angle of 120° and extend over the complete height H_{w} of the angle clamping sleeve 15″. The angle clamping sleeve 15″ furthermore comprises a continuous slit 20″. The angle clamping sleeve 15″ is formed conically tapered over its complete height H_{w}. On the tapered end of the angle clamping sleeve 15″ a partial circumferential crank 28″ is formed. The circumferential crank 28″ is interrupted in a region where the protrusions 40″ extend over the complete height H_{w} of the angle clamping sleeve 15″.

Although the over the complete height H_{w} of the angle clamping sleeve 15″ in this embodiment does not comprise a lateral surface groove or a flap, this does not mean that the angle clamping sleeve 15″ may not be configured accordingly.

As an example, a side view of a cortical screw 41 is described in FIG. 7. The cortical screw 41 comprises a screw head 42 having an external thread 43 and a screw shaft 44 which also comprises an external thread. The external thread of the screw head 42 hereby is finer than the external thread of the screw shaft 44. The screw head is tapered in the direction of the screw shaft 44.

FIG. 8A is a perspective view and FIG. 8B is a top view of an embodiment of a fixing device 100 according the present invention. The fixing device 100 is composed of two tensioning elements 2 according the first embodiment and an angled clamping sleeve 15 according the first embodiment. The two tensioning elements form an aligned region by the face sided receiving section 6a of a first tensioning element 2a and the face sided receiving section 6b of the second tensioning element 2b. The angled clamping sleeve 15 gets inserted into these regions and locks the adjacent tensioning elements 2a, 2b with a predetermined angle a to each other. After the angle locking of the tensioning elements the not shown fixing means can pushed through the receiving sections 6a, 6b and be inserted into the fracture ends of the bone.

The tensioning element 2, 2′, 2″ and the angled clamping sleeve 15, 15′, 15″ are made of biocompatible materials. Further, a hybrid structure of the tensioning element 2, 2′, 2″ is also possible, wherein the wall of the angled sections 8 are composed of a material with the desired elastic properties and further regions of the tensioning element 2, 2′, 2″, for example the receiving sections 6a, 6b are made of a stiff or absorbable material.

As a basic material for the tensioning element 2, 2′, 2″, for instance metals from the group: X42CrMo15, X100CrMo17, X2CrNiMnMoNNb21-16-5-3, X20Cr13, X15Cr13, X30Cr13, X46Cr13, X17CrNi16-2, X14CrMoS17, X30CrMoN15-1, X65CrMo 17-3, X55CrMo14, X90CrMoV18, X50CrMoV15, X 38CrMo V15, G-X 20CrMo13, X39CrMo17-1, X40CrMoVN16-2, X105CrMo17, X20CrNiMoS13-1, X5CrNi18-0, X8CrNiS18-9, X2CrNi19-11, X2CrNi18-9, X10CrNi18-8, X5CrNiMo17-12-2, X2CrNiMo17-12-2, X2CrNiMoN25-7-4, X2CrNiMoN17-13-3, X2CrNiMo17-12-3, X2CrNiMo18-14-3, X2CrNiMo18-15-3; X 2 CrNiMo 18 14 3, X13CrMnMoN18-14-3, X2CrNiMoN22136, X2CrNiMnMoNbN21-9-4-3, X4CrNiMnMo21-9-4, X105CrCoMo18-2, X6CrNiTi18-10, X5CrNiCuNb16-4, X3CrNiCuTiNb12-9, X3CrNiCuTiNb12-9, X7CrNiAl17-7, CoCr20Ni15Mo, G-CoCr29Mo, CoCr20W15Ni, Co-20Cr-15W-10Ni, CoCr28MoNi, CoNi35Cr20Mo10, Ti1, Ti2, Ti3, Ti4, Ti-5Al-2.5Fe, Ti-5Al-2.5Sn, Ti-6Al-4V, Ti-6Al-4V ELI, Ti-3Al-2.5V (Gr9), 99.5Ti, Ti-12Mo-6Zr-2Fe, Ti-13.4Al-29Nb, Ti-13Nb-13Zr, Ti-15Al, Ti-15Mo, Ti-15Mo-5Zr-3Al, Ti-15Sn, Ti-15Zr-4Nb, Ti-15Zr-4Nb-4Ta, Ti-15Zr-4Nb-4Ta-0.2Pd, Ti-29Nb-13Ta-4.6Zr, Ti-30Nb-10Ta-5Zr, Ti-35.5Nb-1.5Ta-7.1Zr, Ti-35Zr-10Nb, Ti-45Nb, Ti-30Nb, Ti-30Ta, Ti-6Mn, Ti-5Zr-3Sn-5Mo-15Nb, Ti-3Al-8V-6Cr-4Zr-4Mo, Ti-6Al-2Nb-1Ta-0.8Mo, Ti-6Al-4Fe, Ti-6Al-4Nb, Ti-6Al-6Nb-1Ta, Ti-6Al-7Nb, Ti-6Al-4Zr-2Sn-2Mo, Ti-8.4Al-15.4Nb, Ti-8Al-7Nb, Ti-8Al-1Mo-1V, Ti-11Mo-6Zr-4Sn, may be considered.

Furthermore, polymers from the group: MBS, PMMI, MABS, CA, CTA, CAB, CAP, COC, PCT, PCTA, PCTG, EVA, EVAL, PTFE, ePTFE, PCTFE, PVDF, PVF, ETFE, ECTFE, FEP, PFA, LCP, PMMA, PMP, PHEMA, Polyamide 66, Polyamide 6, Polyamide 11, Polyamide 2, PAEK, PEEK, PB, PC, PPC, PETP, PBT, MDPE, LDPE, HDPE, UHMWPE, LLDPE, PI, PAI, PEI, PIB, POM, PPO, PPE, PPS, PP, PS, PSU, PESU, PVC, PVC-P, PVC-U, ABS, SAN, TPE-U, TPE-A, TPE-E, PVDC, PVA, SI, PDMS, EPM, EP, UF, MF, PF, PUR, UP, PEBA, PHB, PLA, PLLA, PDLA, PDLLA, PGL, PGLA, PGLLA, PGDLLA, PGL-co-poly TMC, PGL-co-PCL, PDS, PVAL, PCL, Poly-TMC, PUR (linear), NiTi Superelastic, NiTi Shape Memory, may be considered.

Furthermore, also ceramics from the group: Al₂O₃ (alumina oxide), Y-TZP (zirconium oxide ceramic), AMC (alumina matrix composite), HA (hydroxyl apatite), TCP (tricalcium phosphate), Ceravital (glass ceramic/Bioglas®), FZM/K (zirconium oxide, partially stabilized), TZP-A (zirconium oxide ceramic), ATZ (alumina-toughened zirconia), C799 (alumina oxide ceramic), Schott 8625 (transponder glass), may be considered.

Furthermore, also any combinations thereof may be considered.

Apart from the shown embodiments, the invention also allows for further design approaches.

Although the inner surface of the face sided receiving section of the tensioning element or the outer surface of the angle clamping sleeve are formed with tooth systems, the protrusions or recesses can be formed in an arbitrary protruding or recessing way, e.g. nub shaped, as corrugated profile or the like.

Although the angle clamping sleeve above is formed in one piece, it is also possible to form the angle clamping sleeve in multi-piece, e.g. two-piece, three-piece.

The transverse lateral surface groove of the angle clamping sleeve is formed in the embodiments in such a way that the lateral surface groove completely surrounds the angle clamping sleeve; however it is also possible to form the lateral surface groove only in an angle between 90° and 360°. Further, it is not mandatory that the lateral surface groove is directed transversally to the longitudinal axis of the angle clamping sleeve.

The partial circumferential crank of the angle clamping sleeve can be configured in such a way that the crank is flexible and can be adapted to surface of the bone.

Although the screw head of the cortical screw comprises a thread pitch different from the thread pitch of the screw shaft, said thread pitches can be the formed identically.

The teething can be configured as cycloidal teeth, Wildhaber-Novikov teeth, involute toothing or the like.

The above-mentioned tensioning elements as well as the angle clamping sleeves can be manufactured by turning, milling, erosion, injection molding, sintering or the like.

Although above the screw head is formed conically, the screw head can also formed circle-key like, inclined circle-key like, eccentric and so on. In this way oxidation processes or diffusion processes and so on between the screw head and the angle clamping sleeve can be reduced because only a restricted circumferential surface of the screw head contacts the bone plate.

The invention proposes a fixing device for fixing the fracture ends of bones in a bone fracture comprising at least two tensioning elements, each of which has a contoured body that is hollow when viewed from above, has a circumferential wall and two face sided receiving sections lying opposite to each other, at least one clamping sleeve and at least two fixing means which can be position in the face sided receiving sections. The at least two tensioning elements are positioned in relation to each other in such a way that one receiving section of each tensioning element is aligned with the respective other receiving section and are penetrated by the clamping sleeve in such a way that both tensioning elements are coupled to each other. According to invention the clamping sleeve furthermore is an angle clamping sleeve which is connected with the at least two tensioning elements in such a way that the latter lie in a predetermined angular position in relation to each other.

1.-29. (canceled)

30. A fixing device for fixing fracture ends of bones in a bone fracture, wherein the fixing device comprises
at least two tensioning elements, each of which comprising a contoured body which is hollow when viewed from above, has a circumferential wall and two face sided receiving sections lying opposite to each other,
at least one clamping sleeve, and
at least two fixing elements which can be positioned in the face sided receiving sections,
the at least two tensioning elements being positioned in relation to each other in such a way that one receiving section of each tensioning element is aligned with the respective other and are penetrated by the at least one clamping sleeve in such a way that both tensioning elements are coupled to each other,
and wherein the at least one clamping sleeve comprises an angle clamping sleeve that is connected with the at least two tensioning elements in such a way that the latter lie in a predetermined angular position in relation to each other.

31. The fixing device of claim 30, wherein the angle clamping sleeve is connected to the at least two tensioning elements in a form fitting manner.

32. The fixing device of claim 30, wherein on an inner surface of at least one of the face sided receiving sections of a tensioning element at least one protrusion and/or at least one recess is present and on an outer surface of the angle clamping sleeve at least one protrusion and/or at least one recess is present.

33. The fixing device of claim 30, wherein along a inner surface of at least one of the face sided receiving sections of a tensioning element at least in parts internal teeth are present and along an outer surface of the angle clamping sleeve at least in parts external teeth are present.

34. The fixing device of claim 30, wherein at least one face sided receiving section of a tensioning element is tapered.

35. The fixing device of claim 30, wherein the at least two tensioning elements can be coupled to each other via the face sided receiving sections by a tongue and groove connection.

36. The fixing device of claim 30, wherein the angle clamping sleeve is connected to the at least two tensioning elements in a force fitting manner.

37. The fixing device of claim 30, wherein a thread is present on an inner surface of the angle clamping sleeve.

38. The fixing device of claim 30, wherein the angle clamping sleeve is formed from such a material that thread can be screwed into its inner surface by a fixing element.

39. The fixing device of claim 30, wherein an inner surface and/or external teeth of the angle clamping sleeve are at least in parts formed tapered.

40. The fixing device of claim 30, wherein the angle clamping sleeve comprises a continuous slit along its longitudinal axis.

41. The fixing device of claim 30, wherein the angle clamping sleeve comprises at least one non-continuous slit along its longitudinal axis.

42. The fixing device of claim 30, wherein the angle clamping sleeve comprises a lateral surface groove which extends transversally to its longitudinal axis.

43. The fixing device of claim 30, wherein the angle clamping sleeve comprises at least one at least in parts circumferential crank.

44. The fixing device of claim 30, wherein the angle clamping sleeve comprises a flap by which a locking ring retains the angle clamping sleeve with respect to at least one tensioning element.

45. The fixing device of claim 30, wherein the angle clamping sleeve is deformable.

46. The fixing device of claim 30, wherein the angle clamping sleeve is divided in radial direction into two sections and an radial inner section or an radial outer section is made of an absorbable synthetic material.

47. The fixing device of claim 30, wherein fixing elements can be pushed through the angle clamping sleeve.

48. The fixing device of claim 47, wherein the fixing elements comprise a cortical screw.

49. The fixing device of claim 48, wherein the cortical screw comprises a screw head provided with a thread.

50. The fixing device of claim 49, wherein the screw head of the cortical screw comprises a thread pitch different from a thread pitch of a screw shaft.

51. The fixing device of claim 49, wherein the screw head of the cortical screw is formed tapered.

52. A tensioning element suitable for the fixing device of claim 30, wherein the tensioning element comprises a contoured body which is hollow when viewed from above, has a circumferential wall and two face sided receiving sections lying opposite to each other, an inner surface of at least one receiving section being configured in such a way that the tensioning element can be locked in a predetermined angular position with respect to a further tensioning element in a form fitting manner or in force fitting manner.

53. An angle clamping sleeve suitable for the fixing device of claim 30, wherein the angle clamping sleeve comprises elements for establishing a form fit or a force fit at an outer surface so that at least two tensioning elements are lockable in a predetermined angular position.

54. A method for fixing fracture ends of bones in a bone fracture, wherein the method comprises:
(a) positioning at least two tensioning elements in a predetermined angle in such a way that respective receiving sections of the at least two tensioning elements are aligned to each other;
(b) inserting an angle clamping sleeve into an aligned region of the at least two tensioning elements in such a way that a form fitting connection is formed so that the at least two tensioning elements are angle stable with respect to their longitudinal axis;
(c) pushing a fixing element through the angle clamping sleeve; and
(d) inserting the fixing element pushed through the angle clamping sleeve into the bone.

55. The method of claim 54, wherein (c) is carried out prior to (b).

56. The method of claim 54, wherein a deformable angle clamping sleeve is used.
